(19) United States
(12) Patent Application Publication
(10) Pub. No.: US 2022/0288340 A1
(43) Pub. Date: Sep. 15, 2022

(54) MEDICAL TUBE HOLDER SYSTEM AND RELATED METHODS

(71) Applicant: Airway Innovations LLC, Grand Rapids, MI (US)

(72) Inventors: Eric J. VanMiddendorp, Rockford, MI (US); Mark C. Celmer, Saint Joseph, MI (US); Jason L. Barr, Lawton, MI (US); Vaughn R. Gerber, Portage, MI (US); Daniel W. Karadsheh, Zeeland, MI (US); Laure J. Kuipers-Alverson, Holland, MI (US)

(21) Appl. No.: 17/199,039

(22) Filed: Mar. 11, 2021

(51) Int. Cl. A61M 16/06; A61M 16/04

(52) U.S. Cl. CPC A61M 16/0683; A61M 16/0497

(57) ABSTRACT

A medical tube holder comprising a face frame assembly, a strap assembly, a holding assembly and an adapter clip. The face frame assembly has a central member with a base with a first end and a second end, and an attachment rail extending therefrom between the ends. The strap assembly is attachable to the face frame assembly and is configured to extend about the head of a patient so as to secure the face frame assembly. The holding assembly includes a base plate, with an arm extending from a proximal end of the base plate and terminating at a transverse channel. The transverse channel is slidably movable along the attachment rail to attach the holding assembly to the face frame. The adapter clip includes a block member attachable to a medical tube, and, is attached to the holding assembly so as to define a maximum spacing therebetween.

### CROSS-REFERENCE TO RELATED APPLICATION

N/A

### BACKGROUND OF THE DISCLOSURE

### 1. Field of the Disclosure

The disclosure relates in general to medical tube holders, and more particularly, to an endotracheal tube holder that prevents self or accidental extubation.

### 2. Background Art

There is a variety of medical tubing designed for different functions in relation to a patient. One type of medical tubing is an endotracheal tube. An endotracheal tube is configured to be inserted into an oral cavity of a patient and a portion of the airway of the patient. The endotracheal tube is used to provide oxygen or other fluids to the patient. An issue with endotracheal tubes is that they may undergo unplanned extubation, or intentional self-extubation by the patient.

In an unplanned extubation, the endotracheal tube is removed accidentally, for example, during transport of the patient, while rolling the patient over, or via failure of an improperly supported ventilator tube. In self-extubation, a patient removes the endotracheal tube on their own, typically by grabbing and pulling the tube from their oral cavity, by thrashing from side to side or by pushing the tube out with their tongue. It has been discovered that there can be multiple adverse events following unplanned or self extubation, which can result in airway trauma, longer ventilation support durations, longer hospital stays and increased medical costs and increased health risks in general.

Many endotracheal tube supports that are suited to support the endotracheal tube, do not prevent extubation well. Some of these supports include adhesive pads secured to the cheeks of the patient, with a frame secured to the pads to support the endotracheal tube in the patient's mouth. Many times, the adhesive pads cause issues because they do not adhere well, or migrate over time. There also are issues with many endotracheal tube supports causing pressure ulcers on the upper lip because they tend to push the endotracheal tube forcibly against it for long periods of time. Further, with some clamp-like supports, the clamps can become saturated with secretions, thus allowing the endotracheal tube to be easily pulled out.

One solution is disclosed in U.S. Pat. No. 9,981,101 B2 issued to VanMiddendorp et al, the entire specification of which is hereby incorporated by reference in its entirety. This disclosure greatly advances and improves endotracheal tube supports. Nevertheless, there remains room for improving endotracheal tube supports.

### SUMMARY OF THE DISCLOSURE

The disclosure is directed in one aspect to a medical tube holder comprising a face frame assembly, a strap assembly, a holding assembly and an adapter clip. The face frame assembly has a central member with a base positionable so as to be spaced apart from a face of a patient. The central member has a base with a first end and a second end, and an attachment rail extending therefrom between the first end and the second end. The strap assembly is structurally attachable to the face frame assembly. The strap assembly is configured to extend at least partially about the head of a patient so as to secure the face frame assembly in a desired orientation. The holding assembly includes a base plate, with an arm extending from a proximal end of the base plate and terminating at a transverse channel. The transverse channel is slidably movable along the attachment rail of the face frame to attach the holding assembly to the face frame. The adapter clip includes a block member attachable to a medical tube. The adapter clip is attached to the holding assembly so as to define a maximum spacing therebetween.

In some configurations, the attachment rail comprises an axial wall extending from an upper surface of the base. A facing wall is positioned at an outer end of the axial wall.

In some configurations, the attachment rail has a stop member at a first end thereof so as to preclude inadvertent disconnecting of the holding assembly from the attachment rail.

In some configurations, the holding assembly is releasably maintainable in a plurality of positions along the central member of the face frame assembly. For example, the facing wall includes a plurality of notches positioned in a spaced apart relationship between a first end and a second end thereof, and wherein the transverse channel includes an indexing nub structurally configured to engage with at least one of the plurality of notches.

In some configurations, the transverse channel includes a base wall and an outward wall, the outward wall defining a transverse slot. The attachment rail is slidably interfaceable therewith.

In some configurations, the central member further comprises a first head union having an inner end and an outer end. The inner end is attachable to a first end of the central member. An outer end is attachable to the strap assembly. A second head union is provided that has an inner end and an outer end. The inner end is attachable to a second end of the central member and an outer end is attachable to the strap assembly. The first and second head unions facilitate coupling of the central member to the strap assembly.

In some configurations, the block member of the adapter clip further includes an aperture defined therein, with a pair of opposing walls providing ingress into the aperture, the pair of opposing walls engageable with the medical tube and elastically deformable outwardly by the medical tube upon insertion therebetween and direction thereof toward the aperture.

In some configurations, each of the tunnel walls further include an outwardly directed ramp at a distal end thereof.

In some configurations, a side slot is defined in the block member on a side opposite the tunnel walls from the aperture to, in turn, allow for elastic deformation of the tunnel walls.

In some configurations, a locking clasp structurally is configured to extend between the opposing tunnel walls to preclude the passage of the medical tube therebetween.

In some configurations, the locking clasp further comprises a body coupled by a live hinge to a first of the tunnel walls. A locking slot positioned at a distal end of the body, and is configured to releasably retain a second of the tunnel walls.

In some configurations, the holding assembly further includes a first depending flange depending from the base plate at a first side thereof, and a second depending flange depending from the base plate at a second side thereof. In such a configuration, the adapter clip further includes a first side depending wall depending from a first side of the block member and a second side depending wall depending from a second side of the block member. A first slot is defined proximate a first side of the block member. A second slot is defined proximate a second side of the block member. Additionally, a first adapter strap extends from the first depending flange of the holding assembly along an inner surface of the first side depending wall of the adapter clip, through the first slot and over at least a portion of an outer surface of the first side depending wall of the adapter clip, and coupled thereto. And, a second adapter strap extends from the second depending flange of the holding assembly along an inner surface of the second side depending wall of the adapter clip, through the second slot and over at least a portion of an outer surface of the second side depending wall of the adapter clip, and coupled thereto.

In some configurations, the first adapter strap and the second adapter strap each include a plurality of transverse openings therealong spaced apart from each other. The first side depending wall has a locking button extending from the outer surface thereof and the second side depending wall has a locking button extending from the outer surface thereof. Upon positioning, the locking button on the first side depending wall extends through one of the plurality of transverse openings of the first adapter strap and the locking button on the second side depending wall extends through one of the plurality of transverse openings of the second adapter strap.

In some configurations, the first adapter strap and the second adapter strap each include a stop member at a second end thereof. The first depending flange of the holding assembly further includes at least one slot extending therethrough. The stop member of the first adapter strap interfaces with the at least one slot to releasably couple the first adapter strap to the first depending flange. The second depending flange of the holding assembly further includes at least one slot extending therethrough. The stop member of the second adapter strap interfaces with the at least one slot to releasably couple the second adapter strap to the second depending flange.

In some configurations, the holding assembly further includes a fastening system having a slot extending through the base plate on a second side thereof, and, a locking tab extending from the base plate on a first side thereof. A fastening strap member has a first end with an enlarged stop, and a second end, and has a plurality of transverse opening extending therethrough in a spaced apart configuration. The first end of the fastening strap is engageable with the slot of the fastening system. And, the locking tab is extendable through one of the plurality of transverse openings of the fastening strap, to secure a medical tube between the base plate and the strap member.

In some configurations, the strap assembly comprises a central portion, first and second side lower straps, first and second yokes, first overhead strap and a second overhead strap. The first and second side lower straps extend away from the central portion in opposing directions. The first and second lower straps are substantially colinear. The first yoke and second yoke extend in opposite directions from the central portion, and are at least one of perpendicular and oblique to the first and second side lower straps. The first overhead strap extends from the first yoke and is oblique to the first side lower strap. A first side diagonal strap extending from the first yoke and is oblique to the first side lower strap. The first side overhead strap and the first side diagonal strap are one of oblique and perpendicular to the first side lower strap. Similarly, the second overhead strap extends from the second yoke and is oblique to the second side lower strap. The second side diagonal strap extending from the second yoke and is oblique to the second side lower strap. The second side overhead strap and the second side diagonal strap are one of oblique and perpendicular to the second side lower strap.

In some configurations, the medical tube holder further comprises a fastener associated with a distal end of each of the first and second side lower straps, the first and second overhead straps and the first and second side diagonal straps.

In some configurations, the central portion, the first and second side lower straps, the first and second yoke, the first and second overhead straps and the first side and second side diagonal straps are formed from a single substantially planar material.

In another aspect of the disclosure, the disclosure is directed to a medical holder comprising a face frame assembly, a strap assembly, a holding assembly and an adapter clip. The face frame assembly has a central member with a base positionable so as to be spaced apart from a face of a patient. The central member has a base with a first end and a second end. The strap assembly is structurally attachable to the face frame assembly. The strap assembly is configured to extend at least partially about the head of a patient so as to secure the face frame assembly in a desired orientation. The holding assembly includes a base plate, with an arm extending from a proximal end of the base plate and attachable to the face frame. The adapter clip includes a block member attachable to a medical tube. The adapter clip is attached to the holding assembly so as to define a maximum spacing therebetween. The block member of the adapter clip further includes an aperture defined therein, with a pair of opposing walls providing ingress into the aperture. The pair of opposing walls are engageable with the medical tube and elastically deformable outwardly by the medical tube upon insertion therebetween and direction thereof toward the aperture.

In some configurations, the holding assembly further includes a first depending flange depending from the base plate at a first side thereof, and a second depending flange depending from the base plate at a second side thereof. The adapter clip further includes a first side depending wall depending from a first side of the block member and a second side depending wall depending from a second side of the block member. A first slot is defined proximate a first side of the block member and a second slot is defined proximate a second side of the block member. A first adapter strap extends from the first depending flange of the holding assembly along an inner surface of the first side depending wall of the adapter clip, through the first slot and over at least a portion of an outer surface of the first side depending wall of the adapter clip, and coupled thereto. A second adapter strap extends from the second depending flange of the holding assembly along an inner surface of the second side depending wall of the adapter clip, through the second slot and over at least a portion of an outer surface of the second side depending wall of the adapter clip, and coupled thereto.

### BRIEF DESCRIPTION OF THE DRAWINGS

The disclosure will now be described with reference to the drawings wherein:

FIG. 1 of the drawings is a perspective view of a configuration of the medical tube holder system of the present disclosure, showing, in particular, attachment thereof to a patient and also with an endotracheal tube coupled thereto;

FIG. 2 of the drawings is a perspective view of a configuration of the medical tube holder system of the present disclosure, showing, in particular, an endotracheal tube coupled thereto;

FIG. 3 of the drawings is a side elevational view of a configuration of the medical tube holder system of the present disclosure, showing, in particular, an endotracheal tube coupled thereto;

FIG. 4 of the drawings is a top plan view of a configuration of the medical tube holder system of the present disclosure, showing, in particular, an endotracheal tube coupled thereto;

FIG. 5 of the drawings is a perspective view of the face frame assembly of the medical tube holder system of the present disclosure;

FIG. 6 of the drawings is a perspective view of a first head union of the face frame assembly of the medical tube holder system of the present disclosure;

FIG. 7 of the drawings is a perspective view of the central member of the face frame assembly and the holding assembly, showing, in particular, the coupling thereof together;

FIG. 8 of the drawings is a perspective view of the central member of the face frame assembly and the holding assembly, showing, in particular, the coupling thereof together;

FIG. 9 of the drawings is a cross-sectional view of the central member of the face frame assembly and the holding assembly, showing, the coupling of the attachment rail and the transverse channel in slidable engagement;

FIG. 10 of the drawings is a top plan view of a configuration of the strap assembly of the present disclosure;

FIG. 11 of the drawings is a perspective view of the holding assembly, the adapter clip and the adapter straps of the present disclosure with an endotracheal tube coupled thereto;

FIG. 12 of the drawings is a perspective view of the adapter clip of the medical tube holder system of the present disclosure;

FIG. 13 of the drawings is a perspective view of the adapter clip of the medical tube holder system of the present disclosure; and

FIG. 14 of the drawings is a perspective view of the central member of the face frame assembly, the holding assembly, the adapter clip, and adapter strap of the present disclosure, with the endotracheal tube positioned, with the adapter clip positioned to be coupled to the endotracheal tube.

### DETAILED DESCRIPTION OF THE DISCLOSURE

While this disclosure is susceptible of embodiment in many different forms, there is shown in the drawings and described herein in detail a specific embodiment(s) with the understanding that the present disclosure is to be considered as an exemplification and is not intended to be limited to the embodiment(s) illustrated.

It will be understood that like or analogous elements and/or components, referred to herein, may be identified throughout the drawings by like reference characters. In addition, it will be understood that the drawings are merely schematic representations of the invention, and some of the components may have been distorted from actual scale for purposes of pictorial clarity.

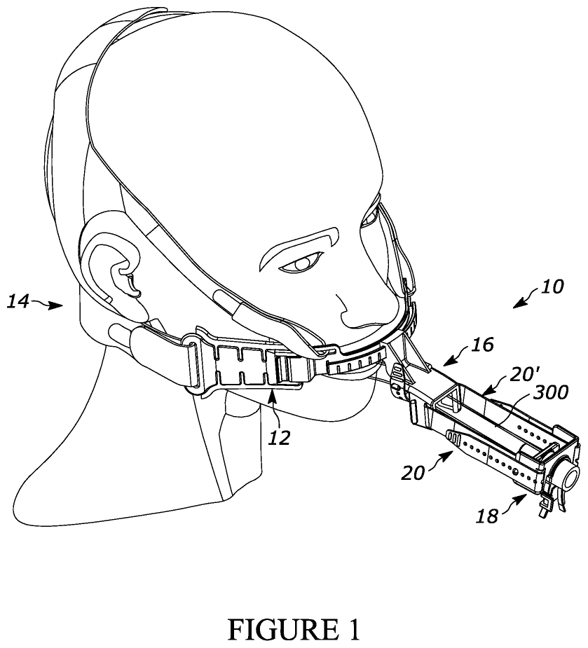

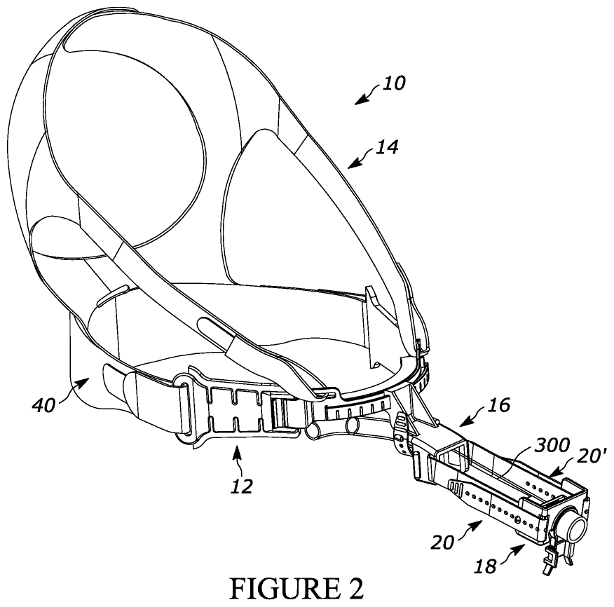

Referring now to the drawings and in particular to FIGS. 1 through 4, collectively, the medical tube holder system is shown generally at 10. The medical tube holder system 10 is configured to retain an endotracheal tube and assembly 300. The endotracheal tube assembly, as will be understood to those of skill in the art, includes a tube 302 having a proximal end and a distal end, wherein the distal end is insertable into the mouth of a user. An inflatable cuff or the like (not shown) may be at the distal end so as to provide a seal with the user. At the proximal end, an end fitting 304 is provided which includes flange 316, an inner tube 318 attachable to the proximal end of the tube 302 and an outer tube that can be coupled to other devices through other hoses and tubes.

The medical tube holder system 10 includes face frame assembly 12, strap assembly 14, holding assembly 16, adapter clip 18 and adapter strap 20. The medical tube holder is configured to preclude extubation that may be inadvertently caused by outside forces, or by the user himself or herself. The medical tube holder precludes such extubation while minimizing discomfort to the user and providing an ease of setup and installation.

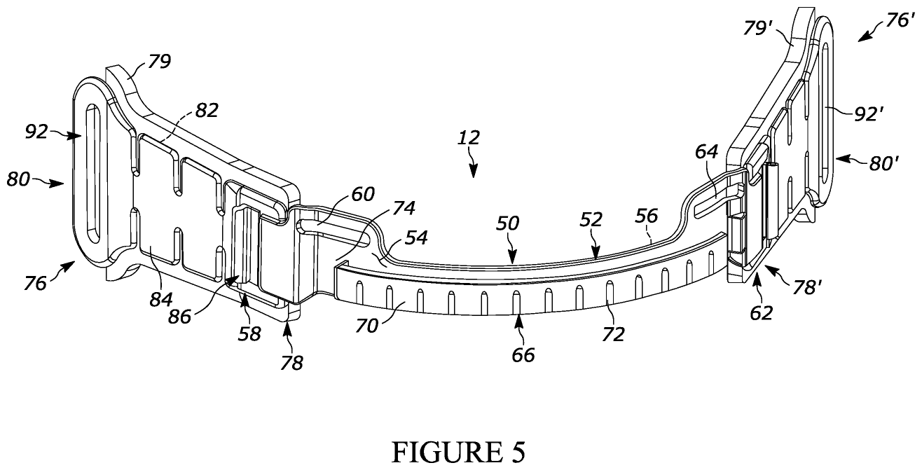

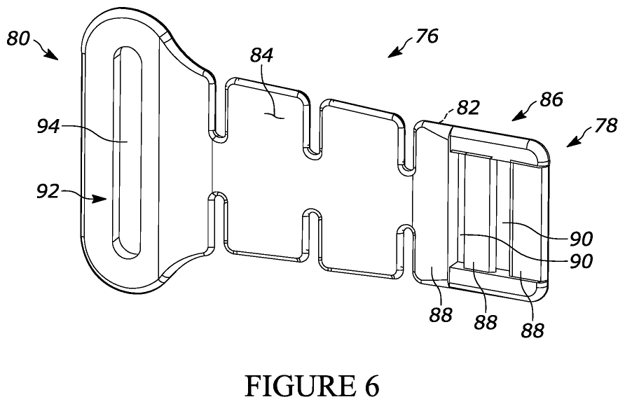

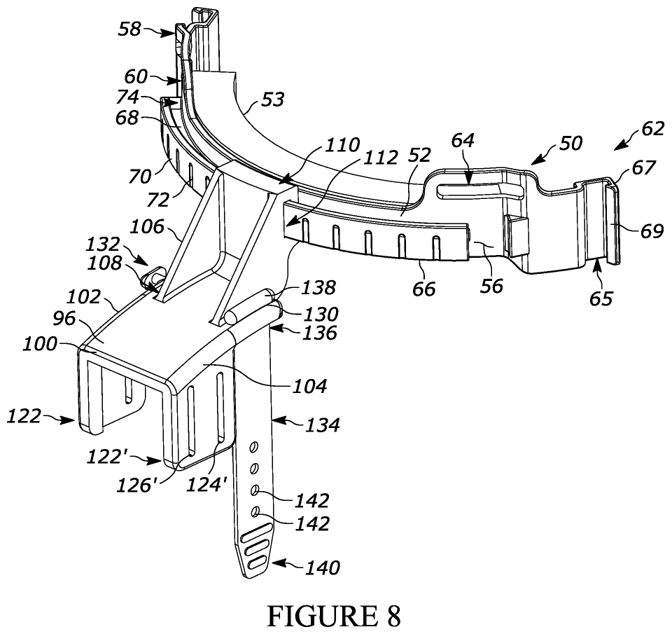

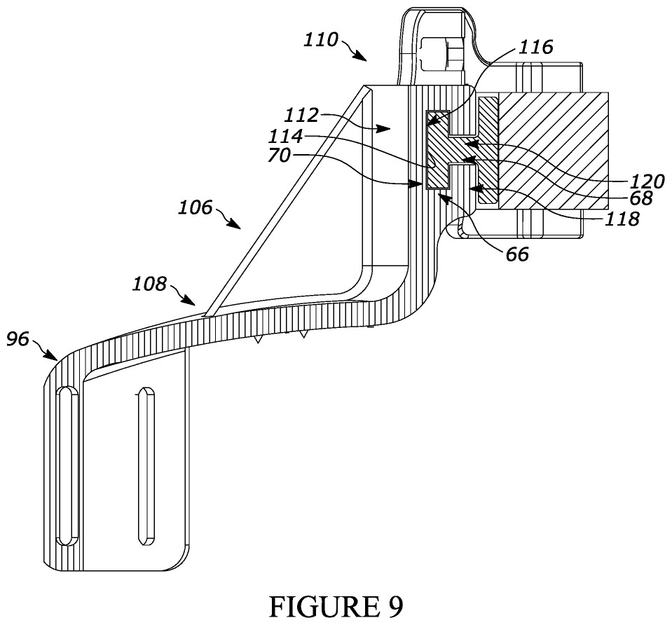

The face frame assembly 12 is shown in greater detail in FIGS. 5 through 9 as comprising central member 50, first head union 76 and second head union 76′. The central member 50 includes base 52 that has an outer surface 54 and an inner surface 56, with the inner surface facing the patient below the nose and above the mouth while being spaced apart therefrom. The base 52 is generally curved so as to substantially track the curved configuration of a face of a user in a spaced apart configuration. In the configuration shown, the base 52 comprises a single radius of curvature, while it is contemplated that the base may include multiple radii of curvature between the first and second end thereof. The base is essentially positionable so as to be spaced apart from a face of a patient. A pad, such as pad 53 may be attached to the inner surface 56.

At a first end of the base, is a first end coupling 58 that is structurally configured to releasably engage the first head union 76 (as will be explained hereinbelow), and first end slot 60 that is structurally configured to engage with the first diagonal strap 38 of the strap assembly 14 (which will also be explained in greater detail below). The first end coupling 58 includes transverse valley 59 terminating with an outward flange 61. The outward flange includes a locking tab 63 at a distal end thereof. The first end slot 58 is generally aligned with the base and substantially perpendicular to the transverse valley 59, as well as being inboard of the first end coupling 58.

At the second end of the base, is a second end coupling 62 which is structurally configured to releasably engage the second head union 76′ and second end slot 64 which is structurally configured to engage with the second diagonal strap 42 of the strap assembly 14. The second end coupling 64 includes transverse valley 65 terminating with an outward flange 67. The outward flange includes a locking tab 69 at a distal end thereof. The second end slot 64 is generally aligned with the base and substantially perpendicular to the transverse valley 65, as well as being inboard of the second end coupling 62.

In the configuration shown, the first end coupling 58 and the first end slot 60 are mirror images of the second end coupling 62 and the second end slot 64 about a midpoint of the base, while variations are contemplated.

The attachment rail 66 of the central member 50 spans from the first end to the second end of the base 52 spaced apart from the first end coupling 58 and the second end coupling 62. In the configuration shown, the attachment rail 66 extends to and overlaps with the first and second end slots 60, 64, and is positioned below the slots. The attachment rail 66 includes axial wall 68 which extends outwardly from the outer surface 54 of the base 52, generally perpendicular thereto and between an upper and lower edge thereof. A facing wall 70 is positioned at the outer end of the axial wall and is generally parallel to and spaced apart from the outer surface 54 of the base 52, thereby defining a substantially “T-shaped” cross-sectional configuration. A plurality of substantially transverse indexing notches, such as indexing notch 72, are positioned in a spaced apart orientation (preferably equally spaced apart from each other and substantially parallel to each other).

As will be explained, these indexing notches are configured to interact with the indexing nub 116 of the arm 106 (it will be understood that the interfacing nub and the index notches may be reversed, in that the attachment rail may include interfacing nubs and an indexing notch may be positioned on the holding assembly), so that the holding assembly is releasably maintainable in a plurality of positions along the central member of the face frame assembly. At a first end of the attachment rail, a stop member 74 is disposed which forms the end of the attachment rail at the first end. As will also be explained below, the arm 106 of the holding assembly 16 is slid into engagement with the second end of the attachment rail and can be disconnected at the second end, while not being allowed to move beyond the stop member 74 at the first end.

The first head union 76 is attached to the first end of the central member 50. The first head union 76 is defined by inner end 78, outer end 80, inner surface 82, outer surface 84. The inner surface faces the user and may include inner pad 79 coupled thereto so as to provide padding between the inner surface and the user's face. Further, an adhesive may be laminated or applied to the pad, and covered with a removable liner. The removable liner can be removed so that the adhesive can secure the pad to the face of the user in the proper orientation. Additionally, the first head union 76 may include a plurality of slots or channels extending therethrough positioned between the first and second ends thereof to allow flexibility and to better follow the contours of the face of the patient.

An inner coupling 86 is structurally configured to couple the first head union 76 to the central member 50. In particular, the inner coupling 86 includes a plurality of traverse bars 88 (in the configuration shown, three spaced apart traverse bars), defining two slots 90 therebetween. To join the inner coupling to the first head union, the first end coupling is threaded between a pair of traverse bars and through the first of the two slots. Subsequently, the first end coupling is threaded between another pair of traverse bars and through the other of the two slots. As the first end coupling passes through the second slot, the central traverse bar is positioned within the transverse valley 59 with the outward flange 61 extending through the second slot, and with the locking tab 63 snapping beyond the slot and into engagement with the central one of the traverse bars, to preclude removal without first manipulating the locking tab 63 away from the central one of the traverse bars.

The first head union 76 further includes an outer coupling 92 structurally configured to engage with the first side lower strap 22 of the strap assembly 14. In the configuration shown, the outer coupling 92 comprises a slot through which the first side lower strap 22 can be directed. In other configurations, the outer coupling may comprise a tab or a nub to which the strap assembly can be coupled.

The second head union 76′ is shown as comprising inner end 78′, outer end 80′, inner surface 82′ and outer surface 84. As with the first head union 76, the second head union 76′ may further include inner pad 79′ positioned on an inner surface 82′ thereof. It will be understood that the second head union 76′ is a substantially mirror image of the first head union 76 and, as such, the first head union was described with the understanding that the second head union has similar if not identical features. Such features have been denoted with the same reference number, augmented by a prime (′). It will be understood that variations may exist between the first and second head unions 76, 76′ on a particular usage, and that such variation is contemplated.

It will be understood that, advantageously, with the use of a first head union and a second head union, in addition to the central member, a single central member may be provisioned, and the first and second head unions may be provided in different sizes so as to accommodate different users. Thus, instead of having a plurality of entire face frame assemblies, the same central member can be utilized across differently sized and shaped faces, with the first and second head unions being provided in a plurality of different sizes. Of course, in other configurations, it is contemplated that the first and second head unions and the central member may be provided as a single integrally molded member, preferably all from a polymer based material. In other configurations, it is contemplated that the central member with a first head union may be provided as a single integrally molded member, and the second head union may be separately provided and attached to the central member opposite the first head unit. Such variations are within the scope of the present disclosure.

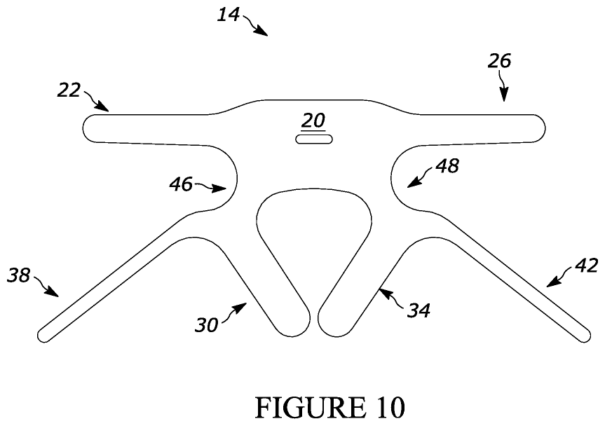

The strap assembly 14, in one configuration, is shown in FIG. 10 as comprising central portion 20 with a first side lower strap 22 and a second side lower strap 26 extending therefrom in opposite directions while being colinear. The strap assembly is configured to extend at least partially about the head of a patient so as to secure the face frame assembly in a desired orientation. A first yoke 46 and second yoke 48 extend away from the central portion 20 one of perpendicular and oblique to each of the respective ones of the first side lower strap and second side lower strap. The central portion 20 may further include a slot, such as slot 21 that is centrally disposed and substantially parallel (while not colinear) with the first and second side lower straps.

The first side overhead strap 30 extends obliquely and inwardly from the first yoke 46. The first side diagonal strap 38 likewise extends from the first yoke 46 obliquely outwardly therefrom. The first side overhead strap 30 and the first side diagonal strap 38 extend one of perpendicularly and obliquely relative to each other away from the first yoke 46. The first side diagonal strap is longer than either one of the first side lower strap and the first side overhead strap.

Similarly, the second side overhead strap 34 extends obliquely and inwardly from the second yoke 48. The second side diagonal strap 42 likewise extends from the second yoke 48 obliquely outwardly therefrom. The second side overhead strap 34 and the second side diagonal strap 42 extend one of perpendicularly and obliquely relative to each other away from the second yoke 46. The second side diagonal strap is longer than either one of the second side lower strap and the second side overhead strap.

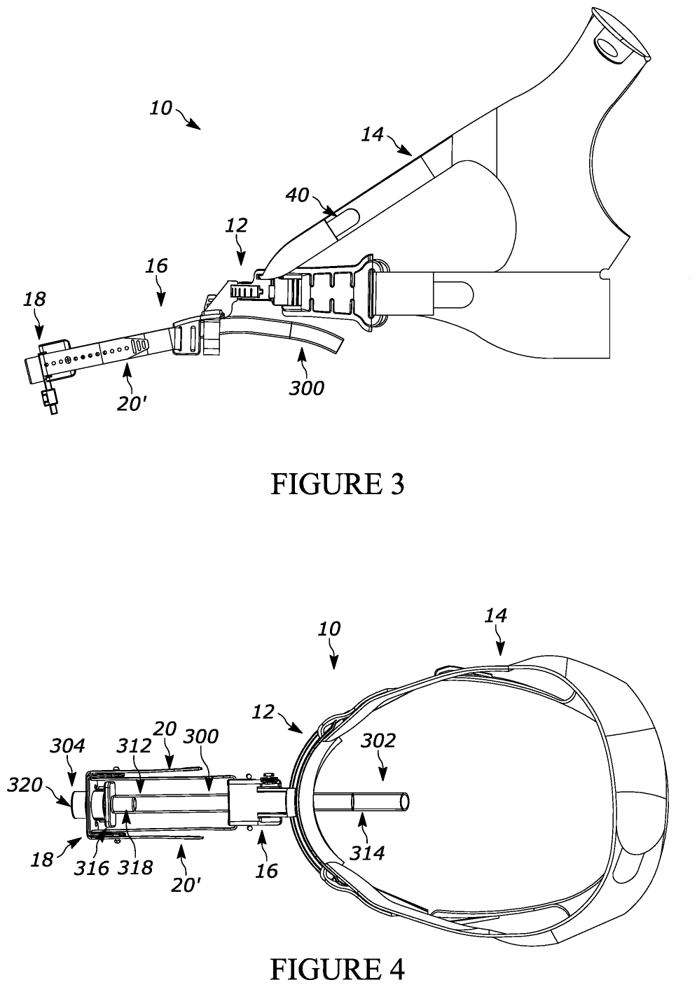

As can be seen in FIG. 10, the strap assembly 14 comprises a single member that is substantially planar in shape and formed from, for example, a neoprene, a fleece material, a woven or nonwoven polymer or other resilient, yet flexible material (i.e., a substantially planar material). In the configuration shown, the first and second sides are mirror images of each other taken about a line that bisects the central portion. Additionally, so as to engage with and to be secured to either itself, another structure, or to the face frame assembly, each of the ends of the strap may include a fastener, including, but not limited to a hook and loop fastener, a snap fit fastener, a tab and slot fastener, a button, among others. In the configuration shown, a plurality of hook and loop fasteners 40 (FIG. 3) are provided. In some configurations, the material from which the strap assembly is formed may comprise the loop of the hook and loop fastener, such than the hook portion may be applied to the particular strap. In the configuration shown, the fasteners are double sided hook tabs so as to minimize stitching in the strap assembly. In other configurations, the tabs may comprise sewed, bonded, or otherwise attached single sided hook, for example, configurations, among others.

As will be explained below, the first side lower strap 22 and second side lower strap 26 are configured to engage and attach to the outer coupling 92, 92′ of the respective one of the first head union 76 and the second head union 76′. The first side diagonal strap 38 and the second side diagonal strap 42 are configured to engage and attach to the respective one of the first end slot 60 and second end slot 64 of the central member 50.

It will be understood that other strap assemblies can be utilized, and the construction is not limited to the strap assembly shown in FIGS. 1-4 and 10.

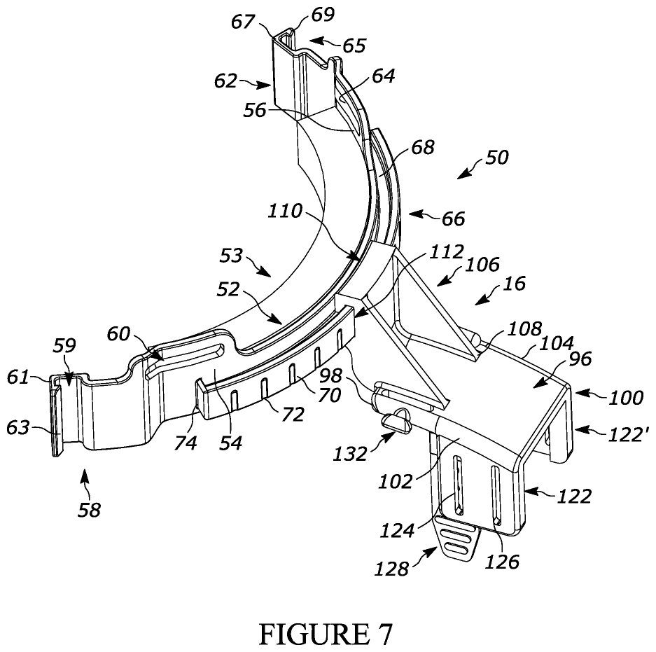

The holding assembly 16 is shown in FIGS. 7, 8 and 11 as comprising base plate 96, arm 106, first depending flange 122, second depending flange 122′, and, fastening system 128. The base plate 96 of the holding assembly 16 comprises a proximal end 98 and a distal end 100, with a first side 102 and a second side 104 opposite the first side. The opposing sides extend substantially from the proximal end to the distal end. The arm 106 extends outwardly and upwardly from the proximal end 98 of the base plate and includes first end 108 and second end 110. A transverse channel 112 is positioned at the second end. The transverse channel 112 includes base wall 114 and outward wall 118. The base wall has an indexing nub 116 extending therefrom. The outward wall 118 includes a transverse slot 120 that extends across the transverse channel 112. The transverse channel 112 substantially matches the attachment rail in cross-sectional configuration, such that the transverse channel 112 of the arm of the holding assembly 16 can be slidingly engaged with the attachment rail. In such a configuration, the indexing nub 116 interfaces with and releasably engages any one of the indexing notches 72 of the attachment rail. It will be understood that the attachment rail and, the transverse channel can each be modified in configuration so that the two structures shape matingly engage with each other.

The first depending flange 122 depends from the first side 102 of the base plate 98. The first depending flange includes an inner slot 124 and an outer slot 126 which are perpendicular to the first side 102 of the base plate and spaced apart from each other. The second depending flange 122′ depends from the second side 104 of the base plate 98. The second depending flange includes an inner slot 124′ and an outer slot 126′ which are perpendicular to the second side 104 of the base plate and spaced apart from each other. In the configuration shown, the inner slots 124, 124′ are opposite each other and substantially correspond. Similarly, outer slots 126, 126′ are opposite each other and substantially correspond.

The fastening system 128 is shown as comprising opposing slots 130, 130′ that extend through the proximal end of the base plate on either side of the first end of arm 106. A locking tab 132 extends outwardly from the first side 102 of the proximal end 98 of the base plate 96. A fastening strap member 134 is provided to extend around the endotracheal tube. The fastening strap member 134 includes a first end 136 having an enlarged stop 138, and a second end 140 with a plurality of transverse openings 142 being positioned in a spaced apart configuration starting proximate the second end 140 and extending toward the first end 136. An adhesive may be applied to the inner surface of the fastening strap member, with a liner positioned over the adhesive prior to use. It is contemplated that in use, the endotracheal tube attaches to the fastening strap member by way of the adhesive.

The fastening strap member is first directed through slot 130′ until the enlarged stop 138 meets the slot 130′ and precludes further passage of the fastening strap through the slot. The fastening strap is then looped around the endotracheal tube and positioned so that one of the transverse openings aligns with the locking tab, and the locking tab is directed through the one of the transverse openings so as to capture the endotracheal tube therebetween. Depending on the size of the endotracheal tube and the patient as well as the position/orientation of the patient relative to the fastening system, a number of transverse openings are provided so as to properly capture the endotracheal tube without imparting undesirable forces thereagainst. The second end can be threaded through the opening 130 so as to be stowed, and to further preclude inadvertent detachment from the locking tab. With the adhesive applied to the fastening strap member, further securement is achieved.

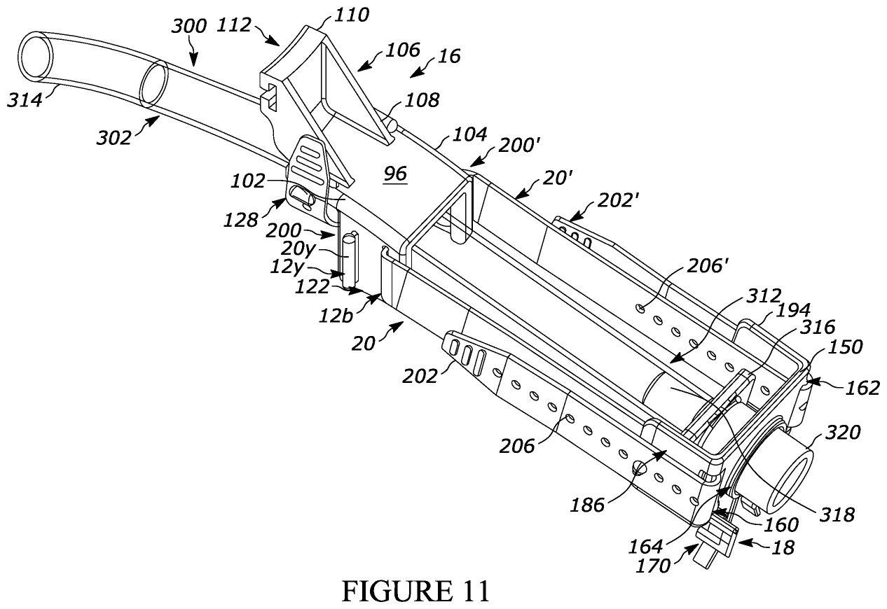

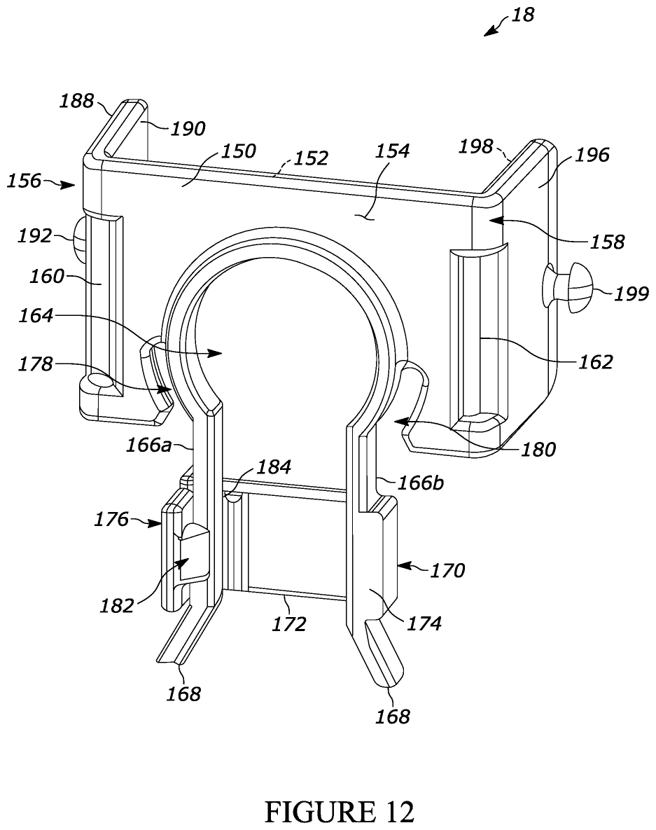

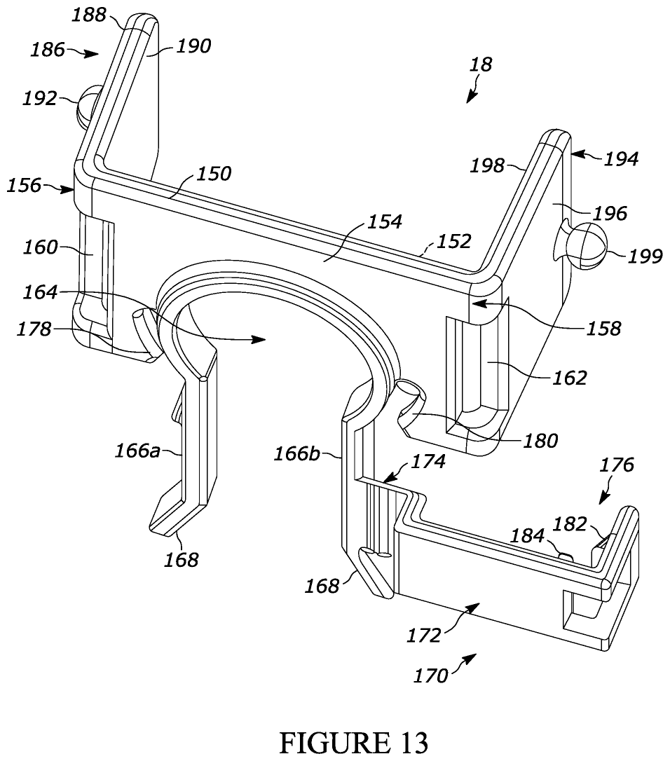

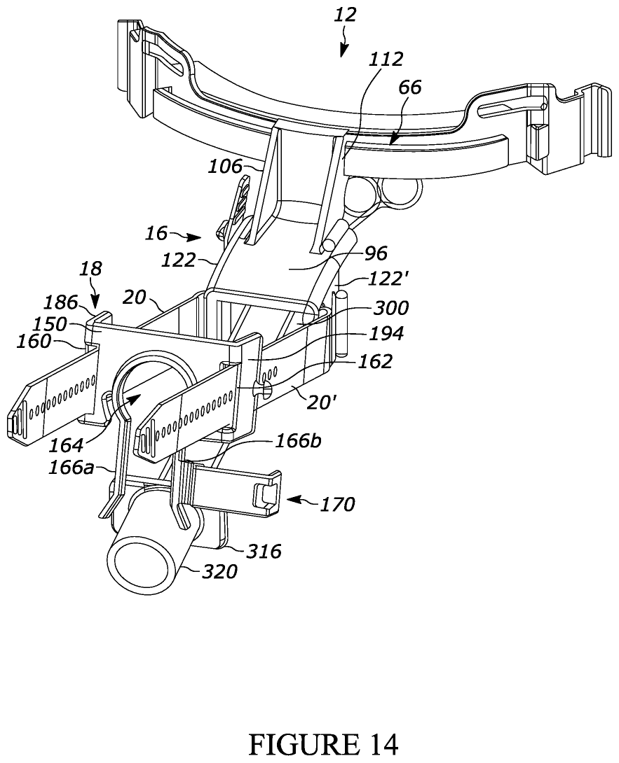

The adapter clip 18 is shown in FIG. 11 through FIG. 14 as comprising block member 150, first side depending wall 186, second side depending wall 194. The block member 150 is defined by inner surface 152, outer surface 154, first side 156, second side 158 and lower end 157. A first side slot 160 is formed where the first side 156 meets the first side depending wall 186. A second side slot 162 is formed where the second side 158 meets the second side depending wall 194. The two slots are generally substantially uniform and mirror images of each other on opposite sides of the block member.

Centrally located on the block member is aperture 164 which substantially matches the outer tuber 320 of the endotracheal tube 300 and terminates proximate lower end 157. A tunnel 166 having opposing tunnel walls 166a, 166b extends from lower end 157 of the aperture 164. Opposing ramps 168, 168′ provide ingress into the tunnel 166 by directing the endotracheal tube toward and into the tunnel 166. The opposing tunnel walls can be elastically deformed outwardly, and side slots 178, 180 provide space for the lower end of the aperture to elastically deform outwardly to accept the endotracheal tube in a snap fit.

A locking clasp 170 may be integrally molded with the block member 150, and may depend from tunnel wall 166b. In particular, such a locking clasp 170 includes body 172, live hinge 174 and locking slot 176. The live hinge is positioned between the tunnel wall 166b and the body 172, with the locking slot 176 formed in the body 172 opposite the live hinge 174. The locking slot 176 includes outer ramp 182 and inner stop 184. It will be understood that to lock the locking clasp, the body 172 is rotated about the live hinge to span between the tunnel walls 166a, 166b. When the body reaches the tunnel wall 166a, the tunnel wall 166a can be elastically deformed to push the outer ramp 182 beyond the tunnel wall 166a at which time the tunnel wall 166a will return to its original position and land within between the inner stop 184 and the outer ramp 182. At such time, the tunnel wall 166a is in locked engagement with the locking clasp. To unlock the locking clasp, the tunnel wall 166 is deflected, elastically inwardly so as to clear the outer ramp 182, at such time, the body 172 can be rotated about the live hinge 174 away from the tunnel wall 166a.

The first side depending wall 186 includes outer surface 188, inner surface 190 and locking button 129 positioned on the outer surface 188. Similarly, the second side depending wall 194 includes outer surface 196, inner surface 198 and locking button 199.

First adapter strap 20 includes first end 200 having stop member 204, and second end 202 spaced apart therefrom. A plurality of openings extend transversely through the first adapter strap 20 starting close to the second end and extending toward the first end in a spaced apart orientation. The second adapter strap 20′ is substantially identical in configuration as the first adapter strap 20, and, as such, the same components have been identified with the same reference number augmented by a prime (′). As will be explained below, the first and second adapter straps 20, 20′ join the adapter clip 18 to the holding assembly 16.

More specifically, the first adapter strap 20 is directed into the inner slot 124 of the first depending flange of the holding assembly 16 and then back through the outer slot 126. Eventually, the stop member 204 reaches the inner slot 124 and precludes further passage of the first adapter strap. The first adapter strap 20 is then directed along the inner surface 190 of the first side depending wall 186 and through the first side slot 160 in the adapter clip. The first adapter strap is then bent around the first depending wall so as to extend along the outer surface 188 thereof. Eventually, when properly positioned, a transverse opening 206 is aligned with locking button 192, at which time, the locking button 192 can be directed through the transverse opening.

Analogously, the second adapter strap 20′ is directed into the inner slot 124′ of the second depending flange of the holding assembly 16 and then back through the outer slot 126′. Eventually, the stop member 204′ reaches the inner slot 124′ and precludes further passage of the second adapter strap. The second adapter strap 20′ is then directed along the inner surface 198 of the second side depending wall 194 and through the second side slot 162 in the adapter clip. The second adapter strap is then bent around the second depending wall so as to extend along the outer surface 196 thereof. Eventually, when properly positioned, a transverse opening 206′ is aligned with a locking button 199, at which time, the locking button 199 can be directed through the transverse opening. In such a configuration the adapter straps maintain the adapter clip at a fixed distance from the holding assembly wherein the adapter straps preclude the adapter clip moving farther away from the holding assembly (i.e., the adapter straps define the maximum spacing between the holding assembly and the adapter clip).

In operation, as a user is intubated, the face frame assembly 12 and the strap assembly 14 can be installed on the head of the user. The steps below are set forth in an order, with the understanding that the assembly and usage may occur in a different order without limitation, and that the order described below is merely exemplary, and not to be deemed limiting. In particular, and utilizing the strap of FIG. 10, the first head union 76 and the second head union 76′ can be joined to opposing ends of the central member 50 to complete the face frame assembly. At the same time (or before, or after), the central portion of the strap assembly can be positioned at the back of the head of the user. Subsequently, the first side overhead strap and the second side overhead strap can be joined together over the top of the user's head. Next, the first side lower strap can be coupled to the outer coupling of the first head union, by extending the end of the strap through the slot 94. Similarly, the second side lower strap can be coupled to the outer coupling of the second head union, by extending the end of the strap through the slot 94′.

Additionally, the first side diagonal strap 38 can be directed through the first end slot 60 of the central member. The second side diagonal strap 42 can be directed through the second end slot 64 of the central member. These can be adjusted and tightened as needed and attached with the fasteners, such as hook and loop fasteners at the ends of each of these straps.

The holding assembly 16 is then coupled to the central member 50 of the face frame 12. Specifically, the arm is positioned proximate the first end of the attachment rail and the transverse channel 112 and the attachment rail are forced into engagement. The transverse channel can then be directed along the attachment rail until centrally positioned, or until in a desired orientation. Generally, as the desired position is reached, the indexing nub 116 will engage one of the indexing notches of the attachment rail so as to releasably lock the two structures together. It will be understood that force is required to remove the indexing nub from within the indexing notches.

With reference to FIGS. 7, 8, 11 and 14, the adapter clip 16 can be attached to the holding assembly 16 through the aforementioned adapter straps 20, 20′. Specifically, the strap 20 interfaces with the first depending flange 122 and the first side depending wall 186. The strap 20′ interfaces with the second depending flange 122′ and the second side depending wall 194.

The endotracheal tube is directed into position between the first depending flange 122 and second depending flange 122′ of the holding assembly 16 and outwardly toward the adapter clip. The endotracheal tube can be coupled to the holding assembly by extending the fastening strap member 134 across from the second depending flange 122′ to the first depending flange 122 wherein the locking tab 132 can be directed through one of the transverse openings 142 of the strap member. The strap member will then maintain the endotracheal tube between the first depending flange 122 and the second depending flange 122′.

The adapter clip 16 can be coupled to the endotracheal tube, and in particular to the outer tube thereof. The adapter clip is rotated (as shown, the adapter clip is positioned over the endotracheal tube) toward the endotracheal tube and into contact thereof. Initially, the ramps 168 interact with the outer tube which outwardly direct the ramps outwardly. As the ramps are spread apart, the outer tube is directed along the opposing tunnel walls 166a, 166b. Eventually, the outer tube reaches the aperture 164 and passes thereinto. Once in the aperture (with which the outer tube shape matingly engages), the opposing tunnel walls 166a, 166b return to their original configuration.

The user can then lock the endotracheal tube within the aperture. In particular, the locking clip can be used to fix the opposing tunnel walls 166a, 166b to each other. Specifically, and as described above, the body 172 can be rotated about the live hinge 174 until the locking slot engages with the tunnel wall 166a at which time, the tunnel wall 166a can be positioned and releasably locked between the outer ramp 182 and the inner stop 184.

With the endotracheal tube interfaced with each of the adapter clip 18 and the holding assembly 16, the endotracheal tube is difficult to extubate inadvertently. Any movement of the endotracheal tube in a direction away from the patient will direct the flange 316 into the block member 150 of the adapter clip 18. And, the adapter clip 18 is precluded from further movement away from the face of the user through the coupling of the adapter straps and the holding assembly.

Rather, the practitioner/user will first have to decouple the endotracheal tube from the adapter clip and then uncouple the fastening strap member 134 from the holding assembly.

The foregoing description merely explains and illustrates the disclosure and the disclosure is not limited thereto except insofar as the appended claims are so limited, as those skilled in the art who have the disclosure before them will be able to make modifications without departing from the scope of the disclosure.

What is claimed is:

1. A medical tube holder comprising:
a face frame assembly having a central member with a base positionable so as to be spaced apart from a face of a patient, the base has a first end and a second end, and an attachment rail extending therefrom between the first end and the second end;
a strap assembly structurally attachable to the face frame assembly, the strap assembly configured to extend at least partially about the head of a patient so as to secure the face frame assembly in a desired orientation;
a holding assembly including a base plate, with an arm extending from a proximal end of the base plate and terminating at a transverse channel, the transverse channel slidably movable along the attachment rail of the face frame to attach the holding assembly to the face frame;
an adapter clip including a block member attachable to a medical tube, the adapter clip being attached to the holding assembly so as to define a maximum spacing therebetween.

2. The medical tube holder of claim 1 wherein the attachment rail comprises an axial wall extending from an upper surface of the base, with a facing wall positioned at an outer end of the axial wall.

3. The medical tube holder of claim 2 wherein the attachment rail has a stop member at a first end thereof so as to preclude inadvertent disconnecting of the holding assembly from the attachment rail.

4. The medical tube holder of claim 2 wherein the holding assembly is releasably maintainable in a plurality of positions along the central member of the face frame assembly.

5. The medical tube holder of claim 2 wherein the transverse channel includes a base wall and an outward wall, the outward wall defining a transverse slot, with the attachment rail slidably interfaceable therewith.

6. The medical tube holder of claim 1 wherein the central member further comprises a first head union having an inner end and an outer end, the inner end attachable to a first end of the central member and an outer end attachable to the strap assembly, and, a second head union having an inner end and an outer end, the inner end attachable to a second end of the central member and an outer end attachable to the strap assembly, the first and second head unions facilitating coupling of the central member to the strap assembly.

7. The medical tube holder of claim 1 wherein the block member of the adapter clip further includes an aperture defined therein, with a pair of opposing walls providing ingress into the aperture, the pair of opposing walls engageable with the medical tube and elastically deformable outwardly by the medical tube upon insertion therebetween and direction thereof toward the aperture.

8. The medical tube holder of claim 7 wherein each of the tunnel walls further include an outwardly directed ramp at a distal end thereof.

9. The medical tube holder of claim 8 wherein a side slot is defined in the block member on a side opposite the tunnel walls from the aperture to, in turn, allow for elastic deformation of the tunnel walls.

10. The medical tube holder of claim 7 further comprising a locking clasp structurally configured to extend between the opposing tunnel walls to preclude the passage of the medical tube therebetween.

11. The medical tube holder of claim 10 wherein the locking clasp further comprises a body coupled by a live hinge to a first of the tunnel walls, and a locking slot positioned at a distal end of the body, the locking slot configured to releasably retain a second of the tunnel walls.

12. The medical tube holder of claim 1 wherein:
the holding assembly further includes a first depending flange depending from the base plate at a first side thereof, and a second depending flange depending from the base plate at a second side thereof;
the adapter clip further includes a first side depending wall depending from a first side of the block member and a second side depending wall depending from a second side of the block member, a first slot defined proximate a first side of the block member and a second slot defined proximate a second side of the block member;
a first adapter strap extending from the first depending flange of the holding assembly along an inner surface of the first side depending wall of the adapter clip, through the first slot and over at least a portion of an outer surface of the first side depending wall of the adapter clip, and coupled thereto; and
a second adapter strap extending from the second depending flange of the holding assembly along an inner surface of the second side depending wall of the adapter clip, through the second slot and over at least a portion of an outer surface of the second side depending wall of the adapter clip, and coupled thereto.

13. The medical tube holder of claim 12 wherein:
the first adapter strap and the second adapter strap each include a plurality of transverse openings therealong spaced apart from each other;
the first side depending wall having a locking button extending from the outer surface thereof and the second side depending wall having a locking button extending from the outer surface thereof; and
wherein, upon positioning, the locking button on the first side depending wall extends through one of the plurality of transverse openings of the first adapter strap and the locking button on the second side depending wall extends through one of the plurality of transverse openings of the second adapter strap.

14. The medical tube holder of claim 13 wherein:
the first adapter strap and the second adapter strap each include a stop member at a second end thereof;
the first depending flange of the holding assembly further including at least one slot extending therethrough, the stop member of the first adapter strap interfacing with the at least one slot to releasably couple the first adapter strap to the first depending flange; and
the second depending flange of the holding assembly further including at least one slot extending therethrough, the stop member of the second adapter strap interfacing with the at least one slot to releasably couple the second adapter strap to the second depending flange.

15. The medical tube holder of claim 1 wherein:
the holding assembly further includes a fastening system having a slot extending through the base plate on a second side thereof, and, a locking tab extending from the base plate on a first side thereof; and
a fastening strap member having a first end with an enlarged stop, and a second end, and having a plurality of transverse opening extending therethrough in a spaced apart configuration, the first end of the fastening strap engageable with the slot of the fastening system, with the locking tab extendable through one of the plurality of transverse openings of the fastening strap, to secure a medical tube between the base plate and the strap member.

16. The medical tube holder of claim 1 wherein the strap assembly comprises:
a central portion;
a first and second side lower straps extending away from the central portion in opposing directions, the first and second lower straps being substantially colinear;
a first yoke and a second yoke extending in opposite directions from the central portion, and being at least one of perpendicular and oblique to the first and second side lower straps;
a first overhead strap extending from the first yoke and being oblique to the first side lower strap, and a first side diagonal strap extending from the first yoke and being oblique to the first side lower strap, the first side overhead strap and the first side diagonal strap being one of oblique and perpendicular to the first side lower strap; and
a second overhead strap extending from the second yoke and being oblique to the second side lower strap, and a second side diagonal strap extending from the second yoke and being oblique to the second side lower strap, the second side overhead strap and the second side diagonal strap being one of oblique and perpendicular to the second side lower strap.

17. The medical tube holder of claim 16 further comprising a fastener associated with a distal end of each of the first and second side lower straps, the first and second overhead straps and the first and second side diagonal straps.

18. The medical tube holder of claim 16 wherein the central portion, the first and second side lower straps, the first and second yoke, the first and second overhead straps and the first side and second side diagonal straps are formed from a single substantially planar material.

19. A medical tube holder comprising:
a face frame assembly having a central member with a base positionable so as to be spaced apart from a face of a patient, the base has a first end and a second end;
a strap assembly structurally attachable to the face frame assembly, the strap assembly configured to extend at least partially about the head of a patient so as to secure the face frame assembly in a desired orientation;
a holding assembly including a base plate, with an arm extending from a proximal end of the base plate and attachable to the face frame;
an adapter clip including a block member attachable to a medical tube, the adapter clip being attached to the holding assembly such so as to define a maximum spacing therebetween, the block member of the adapter clip further includes an aperture defined therein, with a pair of opposing walls providing ingress into the aperture, the pair of opposing walls engageable with the medical tube and elastically deformable outwardly by the medical tube upon insertion therebetween and direction thereof toward the aperture.

20. The medical tube holder of claim 19 wherein:
the holding assembly further includes a first depending flange depending from the base plate at a first side thereof, and a second depending flange depending from the base plate at a second side thereof;
the adapter clip further includes a first side depending wall depending from a first side of the block member and a second side depending wall depending from a second side of the block member, a first slot defined proximate a first side of the block member and a second slot defined proximate a second side of the block member;
a first adapter strap extending from the first depending flange of the holding assembly along an inner surface of the first side depending wall of the adapter clip, through the first slot and over at least a portion of an outer surface of the first side depending wall of the adapter clip, and coupled thereto; and
a second adapter strap extending from the second depending flange of the holding assembly along an inner surface of the second side depending wall of the adapter clip, through the second slot and over at least a portion of an outer surface of the second side depending wall of the adapter clip, and coupled thereto.
